(19) United States
(12) Patent Application Publication
(10) Pub. No.: US 2020/0069359 A1
(43) Pub. Date: Mar. 5, 2020

(54) Electrosurgical Systems and Methods of Configuring the Same

(71) Applicant: Bio-Medical Engineering (HK) Limited, Hong Kong SAR (CN)

(72) Inventors: Chung Kwong Yeung, Hong Kong SAR (CN); Hoi Lam Martin Chow, Hong Kong SAR (CN)

(21) Appl. No.: 16/675,974

(22) Filed: Nov. 6, 2019

Related U.S. Application Data
(63) Continuation of application No. PCT/CN2018/070733, filed on Jan. 3, 2018.

(51) Int. Cl. A61B 18/12

(52) U.S. Cl. CPC A61B 18/1206; A61B 2018/00589

(57) ABSTRACT

Example embodiments relate generally to electrosurgical systems configurable to perform an electrosurgical action (e.g., coagulation) on a target area, and methods of configuring the same. In an exemplary embodiment, the electrosurgical system may comprise an electrosurgical instrument. The electrosurgical instrument may have a first conductive member and a second conductive member. The electrosurgical may further comprise an arrangement of a capacitive element (Cᵣ), a first inductive element (Lᵣ), and a second inductive element (Lₘ). The electrosurgical system may further comprise an input AC voltage source. The input AC voltage source may be configurable top provide an input AC voltage signal. The AC voltage signal may be configurable to have a selected switching frequency (fₛ) and a selected peak voltage value. The switching frequency (fₛ) may be selected based on a desired V-R characteristic for the target area.

### CROSS-REFERENCE TO RELATED APPLICATIONS

This application is a continuation application of International Application No. PCT/CN2018/070733 filed Jan. 3, 2018, the contents of which is hereby expressly incorporated by reference in its entirety, including the contents and teachings of any references contained therein.

### TECHNICAL FIELD

The present disclosure relates generally to electrosurgical systems for use in performing an electrosurgical action, and methods of configuring electrosurgical systems.

### BACKGROUND

In general, electrosurgical systems are used to perform an electrosurgical action by applying electrical power to a portion of a biological tissue. The electrosurgical action may include cutting, coagulating, desiccating, and/or fulgurating of the biological tissue. The biological tissue may be a blood vessel, and luminal structures, etc. Both monopolar and bipolar configurations are commonly used for performing electrosurgical procedures.

### BRIEF SUMMARY

Despite recent developments in modern medical science and technology, it is recognized in the present disclosure that one or more problems are encountered in conventional electrosurgical technologies and methodologies, including those described above and in the present disclosure. For example, conventional electrosurgical systems having bipolar configurations for use in performing coagulation generally require real-time accurate measurements of an impedance (e.g., resistance) of the tissue that is receiving the electrosurgical action so as to enable the electrosurgical system to perform active adjustments of the input received from its power source and the output provided to the tissue via its electrosurgical instruments. Such real-time measurements of the tissue impedance and active adjustments of the input and outputs of the electrosurgical system will require significant processing power and speeds.

Present example embodiments relate generally to and/or comprise systems, subsystems, processors, devices, logic, and methods for addressing conventional problems, including those described above and in the present disclosure, and more specifically, example embodiments relate to electrosurgical systems and methods of configuring the same.

In an exemplary embodiment, an electrosurgical system is described. The electrosurgical system is configurable to perform an electrosurgical action (e.g., coagulation) on a target area (e.g., biological tissue). The electrosurgical system may comprise an electrosurgical instrument. The electrosurgical instrument may have a first conductive member and a second conductive member. The first and second conductive members may be configurable to contact with the target area. The electrosurgical may further comprise an arrangement of a capacitive element (Cᵣ), a first inductive element (Lᵣ), and a second inductive element (Lₘ). The first inductive element (Lᵣ) may be electrically connected to the capacitive element (Cᵣ), and the second inductive element (Lₘ) may be electrically connected to the first inductive element (Lᵣ). The first and second conductive members of the electrosurgical instrument may be electrically connected to the second inductive element (Lₘ) in a parallel arrangement. A resonant frequency (fᵣ) of the arrangement may be based on the capacitive element (Cᵣ) and first inductive element (Lᵣ). The electrosurgical system may further comprise an input AC voltage source. The input AC voltage source may be electrically connected to the arrangement. The input AC voltage source may be configurable to provide an input AC voltage signal. The AC voltage signal may be configurable to have a selected switching frequency (fₛ) and a selected peak voltage value. The switching frequency (fₛ) may be selected based on a desired V-R characteristic for the target area. The switching frequency (fₛ) may be selected as a frequency that may be greater than the resonant frequency (fᵣ) of the arrangement. The desired V-R characteristic may include a plurality of possible electrical resistance values, such as those that may exist for the target area, and a corresponding voltage value to be applied to the target area for each of the possible electrical resistance values. The arrangement may be configured in such a way that when a selected input AC voltage signal having the selected switching frequency (fₛ) and selected peak voltage value may be applied by the input AC voltage source at a time t₁, the arrangement may be configured to apply, via the first and second conductive members of the electrosurgical instrument, an output voltage to the target area that may be based on an electrical resistance of the target area at the time t₁. The arrangement may be further configured in such a way that when the selected input AC voltage signal having the selected switching frequency (fₛ) and selected peak voltage value may continue to be applied by the input AC voltage source after the time t₁, the arrangement may be configured to apply, via the first and second conductive members of the electrosurgical instrument, an output voltage to the target area that adaptively changes in response to changes in electrical resistance of the target area after the time t₁.

In another exemplary embodiment, a method of configuring an electrosurgical system is described. The method may be for use in configuring an electrosurgical system to perform an electrosurgical action (e.g., coagulation) on a target area (e.g., biological tissue). The method may comprise identifying a target area. The method may comprise obtaining a desired V-R characteristic for the target area. The desired V-R characteristic may include a plurality of possible electrical resistance values, such as those that may exist for the target area, and a corresponding voltage value to be applied to the target area for each of the possible electrical resistance values. The method may further comprise configuring an electrosurgical instrument assembly. The electrosurgical instrument assembly may be configured to include an input AC voltage. The electrosurgical instrument assembly be further configured to include an electrosurgical instrument. The electrosurgical system may have a first conductive member and a second conductive member. The electrosurgical may further comprise an arrangement of a capacitive element (Cᵣ), a first inductive element (Lᵣ), and a second inductive element (Lₘ). The first inductive element (Lᵣ) may be electrically connected to the capacitive element (Cᵣ), and the second inductive element (Lₘ) may be electrically connected to the first inductive element (Lᵣ). The first and second conductive members of the electrosurgical instrument may be electrically connected to the second inductive element (Lₘ) in a parallel arrangement. A resonant frequency (fᵣ) of the arrangement may be based on the capacitive element (Cᵣ) and first inductive element (Lᵣ). The method may further comprise selecting an input AC voltage signal. The AC voltage signal is applied by the input AC voltage source. The AC voltage signal may be configurable to have a selected switching frequency (fₛ) and a selected peak voltage value. The switching frequency (fₛ) may be selected based on a desired V-R characteristic for the target area. The switching frequency (fₛ) may be selected as a frequency that may be greater than the resonant frequency (fᵣ) of the arrangement. The method may further comprise contacting the target area between the first and second conductive members of the electrosurgical instrument. The method may further comprise that, while the target area is contacted between the first and second conductive members of the electrosurgical instrument, applying, by the input AC voltage source, the selected input AC voltage signal having the selected switching frequency (fₛ) and selected peak voltage value. The electrosurgical instrument may be configured in such a way that when the selected input AC voltage signal having the selected switching frequency (fₛ) and selected peak voltage value is applied by the input AC voltage source at a time t₁, the electrosurgical instrument assembly may be configured to apply, via the first and second conductive members of the electrosurgical instrument, an output voltage to the target area that may be based on an electrical resistance of the target area at the time t₁. The electrosurgical instrument may be further configured in such a way that when the selected input AC voltage signal having the selected switching frequency (fₛ) and selected peak voltage value continues to be applied by the input AC voltage source after the time t₁, the electrosurgical instrument assembly may be configured to apply, via the first and second conductive members of the electrosurgical instrument, an output voltage to the target area that may adaptively change in response to changes in electrical resistance of the target area after the time t₁.

In another exemplary embodiment, a method of configuring an electrosurgical system is described. The method may include identifying a target area (e.g., biological tissue). The method may also include obtaining a desired V-R characteristic for the target area. The desired V-R characteristic may include a plurality of possible electrical resistance values for the target area and a corresponding voltage value to be applied to the target area for each of the possible electrical resistance values. The method may also include configuring an electrosurgical instrument assembly to include an input AC voltage source configurable to apply an input AC voltage signal. The input AC voltage signal may include a switching frequency (fₛ) and a peak voltage value. The method may also include configuring an electrosurgical instrument assembly to include an electrosurgical instrument having a first conductive member and a second conductive member. The method may also include configuring an electrosurgical instrument assembly to include an arrangement of a capacitive element (Cᵣ) electrically connected to the input AC voltage source, a first inductive element (Lᵣ) electrically connected to the capacitive element (Cᵣ), and a second inductive element (Lₘ) electrically connected to the first inductive element (Lᵣ). The first and second conductive members of the electrosurgical instrument may be electrically connected to the second inductive element (Lₘ) in a parallel arrangement. A resonant frequency (fᵣ) of the arrangement may be based on the capacitive element (Cᵣ) and first inductive element (Lᵣ). The resonant frequency (fᵣ) may be selected based on the desired V-R characteristic for the target area. The resonant frequency (fᵣ) may be selected as a frequency that is less than the switching frequency (fₛ) of the input AC voltage signal. The method may also include contacting the target area between the first and second conductive members of the electrosurgical instrument. While the target area is contacted between the first and second conductive members of the electrosurgical instrument, the method may also include applying, by the input AC voltage source, the input AC voltage signal. The configuring of the electrosurgical instrument assembly may be performed in such a way that, when the input AC voltage signal is applied by the input AC voltage source at a time t₁, the electrosurgical instrument assembly is configured to apply, via the first and second conductive members of the electrosurgical instrument, an output voltage to the target area that is based on an electrical resistance of the target area at the time t₁. The configuring of the electrosurgical instrument assembly may be performed in such a way that, when the input AC voltage signal continues to be applied by the input AC voltage source after the time t₁, the electrosurgical instrument assembly is configured to apply, via the first and second conductive members of the electrosurgical instrument, an output voltage to the target area that adaptively changes in response to changes in electrical resistance of the target area after the time t₁.

### BRIEF DESCRIPTION OF THE DRAWINGS

For a more complete understanding of the present disclosure, example embodiments, and their advantages, reference is now made to the following description taken in conjunction with the accompanying drawings, in which like reference numbers indicate like features, and:

FIG. 1 is a graph that depicts a typical V-R characteristic for a tissue, plotting output voltage power versus tissue resistance;

FIG. 2 is a schematic block diagram representation of an example embodiment of an electrosurgical system according to the present disclosure;

FIG. 3 is a schematic block diagram representation of another example embodiment of an electrosurgical system according to the present disclosure;

FIG. 4 is a graph that depicts various voltage gain-versus-frequency ratio curves under different quality factors;

FIG. 5 is an illustration showing the association of the typical V-R characteristic for a tissue with the various voltage gain-versus-frequency ratio curves; and

FIG. 6 is an illustration of an example embodiment of a method for configuring an electrosurgical system.

Although similar reference numbers may be used to refer to similar elements in the figures for convenience, it can be appreciated that each of the various example embodiments may be considered to be distinct variations.

Example embodiments will now be described with reference to the accompanying drawings, which form a part of the present disclosure and which illustrate example embodiments which may be practiced. As used in the present disclosure and the appended claims, the terms “embodiment,” “example embodiment,” “exemplary embodiment,” and “present embodiment” do not necessarily refer to a single embodiment, although they may, and various example embodiments may be readily combined and/or interchanged without departing from the scope or spirit of example embodiments. Furthermore, the terminology as used in the present disclosure and the appended claims is for the purpose of describing example embodiments only and is not intended to be limitations. In this respect, as used in the present disclosure and the appended claims, the term “in” may include “in” and “on,” and the terms “a,” “an,” and “the” may include singular and plural references. Furthermore, as used in the present disclosure and the appended claims, the term “by” may also mean “from,” depending on the context. Furthermore, as used in the present disclosure and the appended claims, the term “if” may also mean “when” or “upon,” depending on the context. Furthermore, as used in the present disclosure and the appended claims, the words “and/or” may refer to and encompass any and all possible combinations of one or more of the associated listed items.

### DETAILED DESCRIPTION

It is recognized in the present disclosure that one or more problems are encountered in electrosurgical-related technologies and methodologies, including those described above and in the present disclosure. For example, conventional electrosurgical systems having bipolar configurations for performing coagulation on biological tissue require complex processors and/or sensors to perform real-time and accurate measurements of an impedance (e.g., resistance) of a target tissue. The processors of such conventional electrosurgical systems are then required to perform active adjustments of input electrical power (e.g., input AC voltage) so as to produce desired output electrical power (to the electrosurgical instruments of the electrosurgical system), and such active adjustments of the input electrical power are based on the measured tissue impedance. It is recognized in the present disclosure that inaccurate real-time measure of the tissue impedance may result in undesired magnitudes of output electrical power applied to the biological tissue.

Systems, devices, and methods, including those for use in sealing biological tissue, are described in the present disclosure for addressing one or more problems of known systems, devices, and methods, including those described above and in the present disclosure. It is to be understood that the principles described in the present disclosure may be applied outside of the context of electrosurgical systems for sealing biological tissue, such as performing diagnostic procedures, surgical or therapeutic procedures, scientific experiments, and/or other procedures in the same and/or other environments, cavities, and/or organs not described in the present disclosure without departing from the teachings of the present disclosure.

While various embodiments in accordance with the disclosed principles have been described above, it should be understood that they have been presented by way of example only, and are not limiting. Thus, the breadth and scope of the example embodiments described in the present disclosure should not be limited by any of the above-described exemplary embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

Typical V-R Characteristic.

During an electrosurgical action, a surgeon applies an output electrical power via an electrosurgical instrument on a target tissue (e.g., a blood vessel and surrounding tissues). As the electric current is applied to the target tissue, electromagnetic waves resulting from the applied electric current energize the electrons within the tissue. These electrons release energy in the form of heat. As the tissue is heated, collagens and elastin in the tissue denature, resulting in a change in the impendence (e.g., resistance) of the target tissue. As such, during any given electrosurgical procedure, the impedance (e.g., resistance) of the target tissue will change as the output electric power is applied to the target tissue. In this regard, the impedance (e.g., resistance) of a target tissue provides an indication of the degree of desiccation/denaturation of the target tissue.

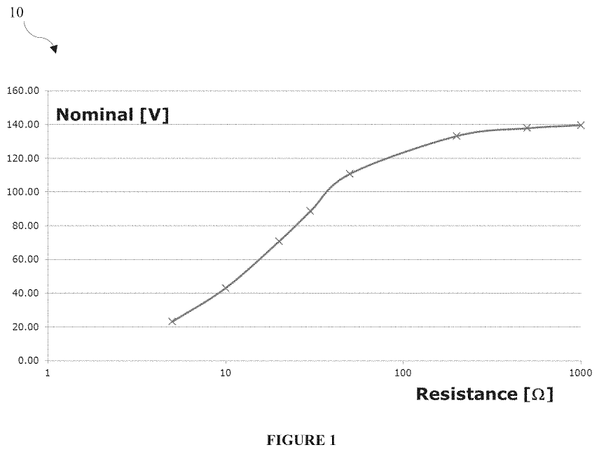

FIG. 1 is an example graph 10 depicting a typical V-R characteristic curve for a target tissue, plotting output voltage power (V) versus tissue resistance (e.g. impedance) (Ω). The x-axis (labelled as “Resistance [Ω]”) represents resistance of the tissue (Ω) and the y-axis (labelled as “Nominal [V]”) represents output voltage (V).

The V-R characteristic curve can be obtained from experimental data, information obtained from historic electrosurgical procedures performed on a given target tissue, etc. A database or a lookup table can be established to store various V-R characteristic curves for different types of tissues.

The electrosurgical system (e.g., electrosurgical system 100).

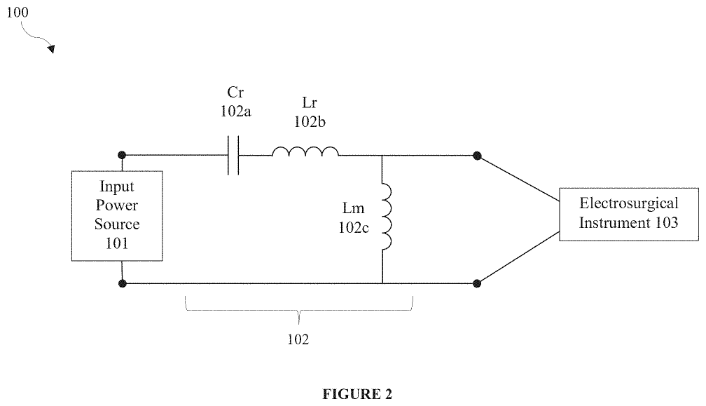

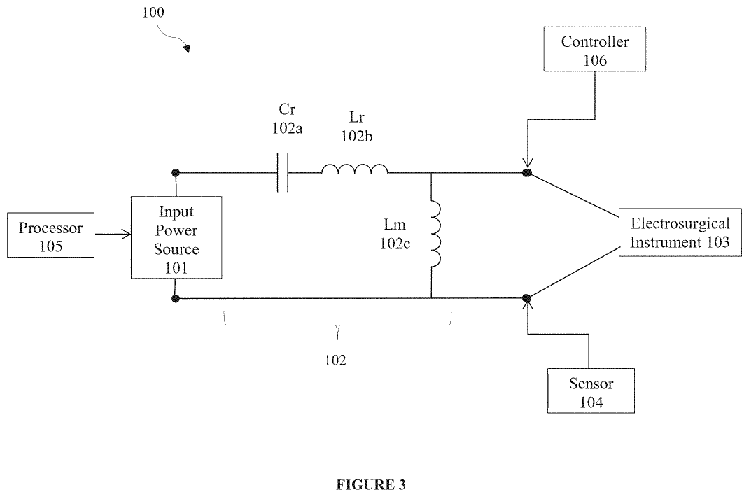

An illustration of example embodiments of an electrosurgical system (or “electrosurgical instrument assembly”) (e.g., electrosurgical system 100) are depicted in FIG. 2 and FIG. 3. The electrosurgical system 100 may include an input power source 101, an LLC resonant circuit assembly 102 having a first element 102a, a second element 102b, a third element 102c, and an electrosurgical instrument 103. These elements of the electrosurgical system 100 will now be further described with reference to the accompanying figures.

The LLC resonant circuit assembly (e.g., LLC resonant circuit assembly 102).

As illustrated in FIGS. 2 and 3, the electrosurgical system 100 may include an LLC resonant circuit assembly (e.g., LLC resonant circuit assembly 102). The LLC resonant circuit assembly 102 may include a first element (e.g., capacitor (Cᵣ) 102a), a second element (e.g., inductor (Lᵣ) 102b), and a third element (e.g., inductor (Lₘ) 102c). In an example embodiment, the first element (e.g., capacitor (Cᵣ) 102a) may be electronically connected to the input power source 101, the second element (e.g., inductor (Lᵣ) 102b) may be electrically connected to the first element (e.g., capacitor (Cᵣ) 102a), and the third element (e.g., inductor (Lₘ) 102c) may be electrically connected to the second element (e.g., inductor (Lᵣ) 102b). The LLC resonant circuit assembly 102 may be configured in such a way as to have a resonant frequency (fᵣ). The resonant frequency (fᵣ) for the LLC resonant circuit assembly 102 may be determined based on the following equation (1):

$${f}_{r} = \frac{1}{2 π \times \sqrt{{L}_{r} \times {C}_{r}}} \qquad Equation ( 1 )$$

Electrosurgical Instrument (e.g., Electrosurgical Instrument 103).

In an example embodiment, the electrosurgical system 100 may include one or more electrosurgical instruments (e.g., electrosurgical instrument 103). For example, as illustrated in FIGS. 2 and 3, the electrosurgical system 100 may include two conductive members 103 (e.g., a sealer, grasper, etc., which may be considered as one electrosurgical instrument 103). The electrosurgical instrument 103 may be configurable or configured to pick, grasp, contact, and/or hold the target tissue between the two conductive members. In example embodiments, the electrosurgical instrument 103 may be configurable or configured to perform a contacting, grasping, sealing, cutting, coagulating, desiccating, and/or fulgurating of the target tissue. Examples of the electrosurgical instrument 103 may include those for use as clamps, forceps, scissors, graspers, etc.

As illustrated in FIGS. 2 and 3, the electrosurgical instrument 103 may be electrically connected to the third element (e.g., inductor (Lₘ) 102c), and such connection may be in a parallel arrangement.

Input Power Source (e.g., 101).

In an example embodiment, the electrosurgical system 100 may include one or more input power sources (e.g., input power source 101). The input power source 101 may be an input AC voltage source. Other types of forms of input power sources are also contemplated without departing from the teachings of the present disclosure.

As illustrated in FIGS. 2 and 3, the input power source 101 may be electrically connected to the LLC resonant circuit assembly 102. Once connected, the input power source 101 may be configurable or configured to supply electrical power to the electrosurgical instrument 103 using or via the LLC resonant circuit assembly 102.

Voltage Gain-Versus-Frequency Ratio Curves.

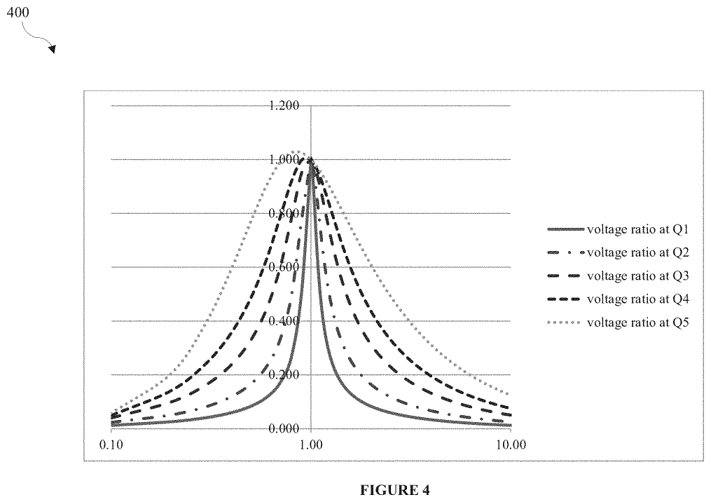

FIG. 4 is an example graph 400 depicting various voltage gain (vertical axis)-versus-frequency ratio (horizontal axis) curves based on different quality factors (Q). FIG. 4 illustrates example curves based on example Q factors Q1, Q2, Q3, Q4, and Q5.

In an example embodiment, the input power source 101 may provide an input voltage (e.g., input AC voltage signal) to the LLC resonant circuit assembly 102, which applies an output voltage to the target tissue via the electrosurgical instrument 103. The input AC voltage signal may have a switching frequency (fₛ) and/or a peak voltage value.

The voltage gain (M) of the LLC resonant circuit assembly 102 may be obtained by:

$$M = \frac{1}{\sqrt{{[ 1 + \frac{1}{m} ( 1 - \frac{1}{{f}^{2}} ) ]}^{2}} + {[ Q ( f - \frac{1}{f} ) ]}^{2}} \qquad Equation ( 2 )$$

Where,

$$Quality factor Q = \frac{\sqrt{{L}_{r} \text{/} {C}_{r}}}{{R}_{tissue}} , \qquad Equation ( 3 )$$

$$\begin{matrix}Normalized frequency ratio f = \frac{{f}_{s}}{{f}_{r}} , & Equation ( 4 ) \\ m = \frac{{L}_{r} + {L}_{m}}{{L}_{r}} , & Equation ( 5 )\end{matrix}$$

Rₜᵢₛₛᵤₑ is the electrical impendence (e.g., resistance) of the target tissue,
fᵣ is the resonant frequency, and
fₛ is the switching frequency.

The voltage gain (M) may be plotted in FIG. 4 for different quality factor (Q) values. In FIG. 4, the x-axis may be a normalized frequency ratio Fₓ, the y-axis may be the voltage gain M. The voltage gain M value may be obtained by the above Equation (2).

Table 1 provides an example of some of the values that can be used to plot the voltage gain curve.

TABLE 1

| Q | Lᵣ (H) | Cᵣ (F) | Lₘ (H) | Frequency Ratio f (fₛ/fᵣ) |
|---|---|---|---|---|
| Q1 = 8.00 | 2.72E−06 | 1.13E−07 | 1.36E−05 | selected from 0.1~10 |
| Q2 = 4.00 | 2.72E−06 | 1.13E−07 | 1.36E−05 | selected from 0.1~10 |
| Q3 = 2.00 | 2.72E−06 | 1.13E−07 | 1.36E−05 | selected from 0.1~10 |
| Q4 = 1.33 | 2.72E−06 | 1.13E−07 | 1.36E−05 | selected from 0.1~10 |
| Q5 = 0.8 | 2.72E−06 | 1.13E−07 | 1.36E−05 | selected from 0.1~10 |

Association of the Typical V-R Characteristic with the Voltage Gain-Versus-Frequency Ratio Curves.

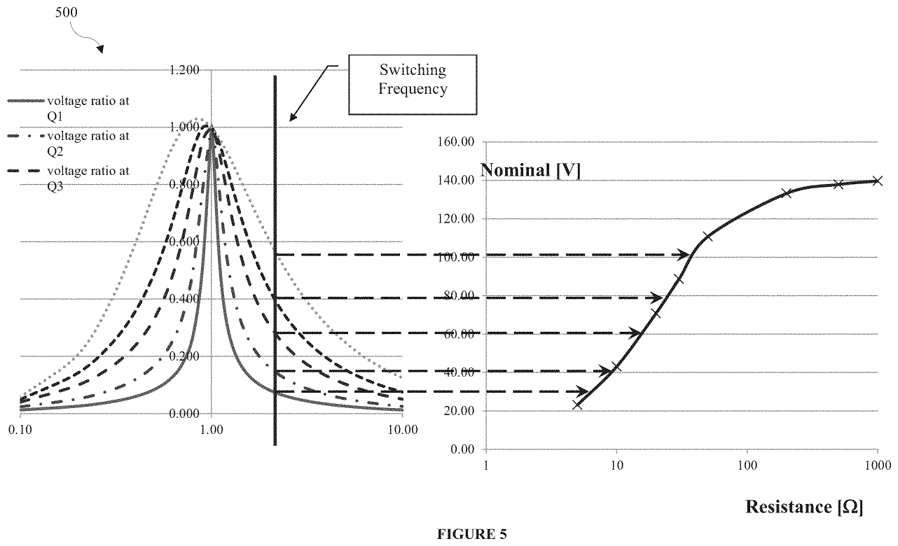

FIG. 5 is an example illustration of a method 500 of associating a V-R characteristic graph (graph on the right hand side of FIG. 5, similar to the graph illustrated in FIG. 1) for a tissue with one or more voltage gain-versus-frequency ratio curves (graph on the left hand side, similar to the graph illustrated in FIG. 4).

In an example embodiment, a desired switching frequency (fₛ) may be selected for the input AC voltage signal of the input power source 101. The switching frequency (fₛ) may be greater than the resonant frequency (fᵣ) of the LLC resonant circuit assembly 102 in example embodiments. For example, the resonant frequency (fᵣ) may be less than or equal to about 1000 kHz and the switching frequency (fₛ) may be less than or equal to about 2000 kHz. In another example embodiment, a desired resonant frequency (fᵣ) may be selected for the arrangement (e.g., LLC resonant circuit assembly 102). The desired resonant frequency (fᵣ) may be less than the switching frequency (fₛ) of the input AC voltage signal. For example, the first element (e.g., capacitor (Cᵣ) 102a) may be less than or equal to about 10 F, the second element (e.g., inductor (Lᵣ) 102b) may be less than or equal to about 10 H, and the third element (e.g., inductor (Lₘ) 102c) may be less than or equal to about 10 H.

In an example embodiment, the ratio of the switching frequency (fₛ) to resonant frequency (fᵣ) may be between about 1:1 and 2:1.

As illustrated in FIG. 5, in example embodiment, the desired V-R characteristics of a target tissue may be mapped or related to the voltage gain-versus-frequency ratio curves (graph on the left hand side) based on the desired switching frequency (fₛ) (as illustrated in FIG. 5) and/or the desired resonant frequency (fᵣ), thereby enabling the system 100 to automatically provide specific output voltages (as illustrated in the graph on the right hand side) based on the impedance (e.g., resistance) of the target tissue and without the need to actively adjust the input AC voltage values.

By way of example, it is recognized that the impedance of a target tissue may change during an electrosurgical procedure of applying an output voltage to the target tissue via the electrosurgical instruments (e.g., when sealing a blood vessel). In this regard, example embodiments of the system 100 are configurable or configured to change the voltage gain-versus-frequency ratio curves (graphs on the left hand side) in response to the change of tissue impedance so as to provide an output voltage corresponding to the tissue impedance. Put differently, the output voltage changes based on the current tissue impedance and without the need to actively adjust the input AC voltage values.

Certain considerations may be taken into account when selecting a desired switching frequency (fₛ) and/or the desired resonant frequency (fᵣ). In an example embodiment, the desired switching frequency (fₛ) and/or the desired resonant frequency (fᵣ) may be selected based on the desired V-R characteristic for the target tissue. Alternatively or in addition, the desired switching frequency (fₛ) and/or the desired resonant frequency (fᵣ) may be selected in such a way as to minimize a difference between the output voltage that should be applied to the target tissue for a particular resistance value pursuant to the desired V-R characteristic and an actual output voltage applied to the target tissue.

According to the present disclosure, when the input AC voltage signal having the switching frequency (fₛ) and/or peak voltage value is applied by the input power source 101 at a time t₁, the electrosurgical system 100 may be configurable or configured to apply, via the first and second conductive members of the electrosurgical instrument 103, an output voltage to the target tissue (or target area) that is based on an impedance (e.g., electrical resistance) of the target tissue at the time t₁. Furthermore, when the input AC voltage signal having switching frequency (fₛ) and/or peak voltage value continues to be applied by the input power source 101 after the time t₁, the electrosurgical system 100 may be configurable or configured to apply, via the first and second conductive members of the electrosurgical instrument 103, an output voltage to the target tissue that adaptively changes in response to changes in electrical resistance of the target tissue after the time t₁.

According to the present disclosure, the output voltage applied, via the first and second conductive members of the electrosurgical instrument 103, to the target tissue after the time t₁ adaptively changes solely in response to changes in electrical resistance of the target tissue after the time t₁ and without requiring any change to the switching frequency (fₛ) and/or peak voltage value of the input AC voltage signal.

According to the present disclosure, changes in electrical resistance of the target tissue after the time t₁ may be caused by the applying of the input AC voltage signal. It is recognized in the present disclosure that the adaptive changing of the output voltage applied, via the first and second conductive members of the electrosurgical instrument 103, to the target area after the time t₁, may be automatically achieved without any measuring of the actual electrical resistance of the target tissue.

Processor (e.g., Processor 105).

In an example embodiment, the electrosurgical system 100 may include a processor (e.g., processor 105) (as illustrated in FIG. 3). The processor 105 may be configurable or configured to receive and/or store the desired V-R characteristic for the target tissue. The processor may also be configurable or configured to select the switching frequency (fₛ) and/or the resonant frequency (fᵣ) based on the received desired V-R characteristic for the target tissue.

In another example embodiment, the processor 105 may be configurable or configured to associate the actual output voltage applied to the target area with a particular voltage pursuant to the desired V-R characteristic to determine a status of the target tissue.

In another example embodiment, the processor 105 may be configurable or configured to terminate the applying of the input AC voltage signal, by the input power source 101, when the actual output voltage applied to the target tissue reaches a maximum voltage value pursuant to the desired V-R characteristic.

Sensor (e.g., Sensor 104).

In an example embodiment, the electrosurgical system 100 may include one or more sensors (e.g., sensor 104) (as illustrated in FIG. 3). The sensor 104 may be configurable or configured to sense one or more parameters of an operating environment. The one or more parameters may include, but are not limited to, temperature, applied pressure, gas formation, or a combination thereof. The one or more parameters of an operating environment may be sensed so as to avoid excess electrical power to be applied onto the target tissue.

Controller (e.g., Controller 106).

In an example embodiment, the electrosurgical system 100 may include a controller (e.g., controller 106) (as illustrated in FIG. 3). The controller 106 may be configurable or Configured to (1) monitor the output voltage applied to the target area and/or the electrical resistance of the target area, and/or (2) provide control and/or feedback signals to the input AC voltage source.

Method of Configuring an Electrosurgical System (e.g., Method 600).

Example embodiments of the electrosurgical system 100 may be configurable or configured to perform an electrosurgical action (e.g., sealing of a blood vessel) in one or more of a plurality of ways.

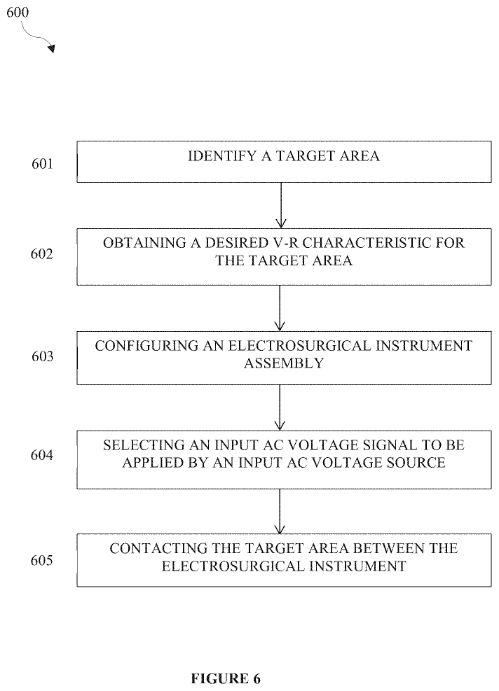

As illustrated in FIG. 6, an example embodiment of a method (e.g., method 600) of configuring an electrosurgical system (e.g., electrosurgical system 100) may include identifying a target tissue (or target area) (e.g., action 601). The method 600 may also include obtaining a desired V-R characteristic for the target tissue (e.g., action 602). The method 600 may also include configuring an electrosurgical system (e.g., electrosurgical system 100) (e.g., action 603). The method 600 may also include selecting an input AC voltage signal to be applied by an input AC voltage source (e.g., input AC voltage source 101) (e.g., action 604). The method 600 may also include contacting the target tissue between the electrosurgical instrument (e.g., electrosurgical instrument 103) (e.g., action 605).

Example embodiments of the method 600 may include or not include one or more of the actions described above and in the present disclosure, may include additional actions, operations, and/or functionality, may be performed in different sequences and/or combinations, and/or one or more of the actions, operations, and/or functionality may be combinable into a single action, operation, and/or functionality and/or divided into two or more actions, operations, and/or functionalities. The method 600, and elements and functionality thereof, will now be further explained with reference to the accompanying figures.

Identifying a Target Area (e.g., Action 601).

In an example embodiment, during an electrosurgical action, a surgeon may firstly identify a target area (or target tissue) to which the surgical action is to be performed (e.g., action 601). The target area may be a gastrointestinal site or an abdominal site. The target area may be a biological tissue, such as a blood vessel.

Obtaining a Desired V-R Characteristic for the Target Area (e.g., Action 602).

In an example embodiment, based on the target area identified, the method 600 may also include obtaining a desired V-R characteristic for the target area (e.g., action 602). The desired V-R characteristic may include a plurality of possible electrical resistance value for each different target area, and a corresponding voltage value that should be applied to the target area for each of the possible electrical resistance values.

The V-R characteristic may be a representation of a relationship between the output voltage power (V) and the tissue impedance (a). The V-R characteristic may also be a representation of an indication of the degree of desiccation/denaturation of a target tissue.

The V-R characteristic curve can be obtained from experimental data, historic information from past surgical procedures, etc. A database or a lookup table can be established to store various V-R characteristic curves for different types of tissues.

Configuring an Electrosurgical Instrument Assembly (e.g., Action 603).

In an example embodiment, the method 600 may also include configuring an electrosurgical system (e.g., electrosurgical system 100) (e.g., action 603). The electrosurgical system 100 may include an input AC voltage source (e.g., input power source 101). The electrosurgical system (e.g., electrosurgical system 100) may also include an electrosurgical instrument (e.g., electrosurgical instrument 103) having a first conductive member and a second conductive member. The electrosurgical system (e.g., electrosurgical system 100) may also include an arrangement (e.g., LLC resonant circuit assembly 102) electrically connected to the input power source (e.g., input AC voltage source 101). The arrangement (e.g., LLC resonant circuit assembly 102) may include a first element (e.g., capacitor (Cᵣ) 102a) electrically connected to the input power source (e.g., input AC voltage source 101). The arrangement (e.g., LLC resonant circuit assembly 102) may also include a second element (e.g., inductor (Lᵣ) 102b) electrically connected to the first element (e.g., capacitor (Cᵣ) 102a). The arrangement (e.g., LLC resonant circuit assembly 102) may also include a third element (e.g., inductor (Lₘ) 102c) electrically connected to the second element (e.g., inductor (Lᵣ) 102b). The first and second conductive members of the electrosurgical instrument (e.g., electrosurgical instrument 103) may be electrically connected to the third element (e.g., inductor (Lₘ) 102c) in a parallel arrangement. A resonant frequency (fᵣ) of the arrangement (e.g., LLC resonant circuit assembly 102) may be based on the first element (e.g., capacitor (Cᵣ) 102a) and the second element (e.g., inductor (Lᵣ) 102b).

In another example embodiment, the electrosurgical instrument assembly may be configured in such a way that the resonant frequency (fᵣ) of the arrangement (e.g., LLC resonant circuit assembly 102) may be selected based on the desired V-R characteristic for the target area. The resonant frequency (fᵣ) may be selected as a frequency that is less than the switching frequency (fₛ) of the input AC voltage signal.

For example, the first element (e.g., capacitor (Cᵣ) 102a) may be selected to be less than or equal to about 10 F. Furthermore, the second element (e.g., inductor (Lᵣ) 102b) may be selected to be less than or equal to about 10 H. Furthermore, the third element (e.g., inductor (Lₘ) 102c) may be selected to be less than or equal to about 10 H.

Selecting an Input AC Voltage Signal to be Applied by an Input AC Voltage Source (e.g., Action 604).

In an example embodiment, the method 600 may include selecting an input AC voltage signal to be applied by the input power source (e.g., input AC voltage source 101) (e.g., action 604). The selected input AC voltage signal may include a selected switching frequency (fₛ) and/or selected peak voltage value. The switching frequency (fₛ) may be selected based on the desired V-R characteristic for the target area. The switching frequency (fₛ) may be selected as a frequency that is greater than the resonant frequency (fᵣ) of the arrangement (e.g., LLC resonant circuit assembly 102).

As described above and in the present disclosure, when selecting a desired switching frequency (fₛ) and/or a desired resonant frequency (fᵣ), one or more conditions may be taken into consideration. For example, the desired switching frequency (fₛ) and/or the desired resonant frequency (fᵣ) may be selected based on the desired V-R characteristic for the target tissue. As another example, the desired switching frequency (fₛ) and/or the desired resonant frequency (fᵣ) may be selected in such a way as to minimize a difference between the output voltage that should be applied to the target tissue for a particular resistance value pursuant to the desired V-R characteristic and an actual output voltage applied to the target tissue.

In another example embodiment, the switching frequency (fₛ) and/or the desired resonant frequency (fᵣ) may be selected in such a way that a ratio of the switching frequency (fₛ) to resonant frequency (fᵣ) is between 1:1 and 2:1. Furthermore, the resonant frequency (fᵣ) may be selected between about 0 kHz to 1000 kHz and the switching frequency (fₛ) is between about 0 kHz to 2000 kHz.

Contacting the Target Area Between the Electrosurgical Instrument (e.g., Action 605).

In an example embodiment, the method 600 may include contacting the target area (or target tissue) between the first and second conductive members of the electrosurgical instrument (e.g., electro surgical instrument 103) (e.g., action 605). While the target area is contacted between the first and second conductive members of the electrosurgical instrument (e.g., electrosurgical instrument 103), the method 600 may include applying, by the input power source (e.g., input AC voltage source 101), the input AC voltage signal having the switching frequency (fₛ) and/or peak voltage value (e.g., as selected in action 604).

In another example embodiment, the configuring of the electrosurgical system 100 may be performed in such a way that when the input AC voltage signal having the switching frequency (fₛ) and/or peak voltage value is applied by the input power source (e.g., input AC voltage source 101) at a time t₁, the electrosurgical system may be configurable or configured to apply, via the first and second conductive members of the electrosurgical instrument (e.g., electrosurgical instrument 103), an output voltage to the target area that is based on an electrical resistance of the target area at the time t₁. Furthermore, when the input AC voltage signal having the switching frequency (fₛ) and/or peak voltage value continues to be applied by the input power source (e.g., input AC voltage source 101) after the time t₁, the electrosurgical system (e.g., electrosurgical system 100) may be configurable or configured to apply, via the first and second conductive members of the electrosurgical instrument (e.g., electrosurgical instrument 103), an output voltage to the target area that adaptively changes in response to changes in electrical resistance of the target area after the time t₁.

In another example embodiment, the output voltage applied, via the first and second conductive members of the electrosurgical instrument (e.g., electrosurgical instrument 103), to the target area after the time t₁ may adaptively change solely in response to changes in electrical resistance of the target area after the time t₁ and without requiring any change to the switching frequency (fₛ) and/or peak voltage value of the input AC voltage signal.

In another example embodiment, the changes in electrical resistance of the target area after the time t₁ may be caused by the applying of the input AC voltage signal. Furthermore, the adaptive changing of the output voltage applied, via the first and second conductive members of the electrosurgical instrument (e.g., electrosurgical instrument 103), to the target area after the time t₁, may be automatically achieved without any measuring of the actual electrical resistance of the target area.

In another example embodiment, the output voltage applied, via the first and second conductive members of the electrosurgical instrument (e.g., electrosurgical instrument 103), to the target area after the time t₁ may adaptively change solely in response to changes in electrical resistance of the target area after the time t₁ and without requiring any change to the arrangement.

In another example embodiment, the method 600 may also include associating the actual output voltage applied to the target area with a particular voltage pursuant to the desired V-R characteristic to determine a status of the target area.

In another example embodiment, the method 600 may also include terminating, by the input power source (e.g., input AC voltage source 101), the applying of the input AC voltage signal when the actual output voltage applied to the target area reaches a maximum voltage value pursuant to the desired V-R characteristic.

In another example embodiment, the method 600 may also include sensing one or more parameters of an operating environment. The one or more parameters may include temperature, applied pressure, gas formation, or a combination thereof. The one or more parameters of an operating environment may be sensed so as to avoid excess electrical power to be applied onto the target tissue.

Various terms used herein have special meanings within the present technical field. Whether a particular term should be construed as such a “term of art” depends on the context in which that term is used. Such terms are to be construed in light of the context in which they are used in the present disclosure and as one of ordinary skill in the art would understand those terms in the disclosed context. The above definitions are not exclusive of other meanings that might be imparted to those terms based on the disclosed context.

Words of comparison, measurement, and timing such as “at the time,” “equivalent,” “during,” “complete,” and the like should be understood to mean “substantially at the time,” “substantially equivalent,” “substantially during,” “substantially complete,” etc., where “substantially” means that such comparisons, measurements, and timings are practicable to accomplish the implicitly or expressly stated desired result.

Additionally, the section headings and topic headings herein are provided for consistency with the suggestions under various patent regulations and practice, or otherwise to provide organizational cues. These headings shall not limit or characterize the embodiments set out in any claims that may issue from this disclosure. Specifically, a description of a technology in the “Background” is not to be construed as an admission that technology is prior art to any embodiments in this disclosure. Furthermore, any reference in this disclosure to “disclosure” in the singular should not be used to argue that there is only a single point of novelty in this disclosure. Multiple disclosures may be set forth according to the limitations of the claims issuing from this disclosure, and such claims accordingly define the disclosure(s), and their equivalents, that are protected thereby. In all instances, the scope of such claims shall be considered on their own merits in light of this disclosure, but should not be constrained by the headings herein.

What is claimed is:

1. A method of configuring an electrosurgical system, the method comprising:
identifying a target area;
obtaining a desired V-R characteristic for the target area, the desired V-R characteristic including a plurality of possible electrical resistance values for the target area and a corresponding voltage value to be applied to the target area for each of the possible electrical resistance values;
configuring an electrosurgical instrument assembly, the electrosurgical instrument assembly configured to include:
an input AC voltage source;
an electrosurgical instrument having a first conductive member and a second conductive member; and
an arrangement of a capacitive element (Cᵣ) electrically connected to the input AC voltage source, a first inductive element (Lᵣ) electrically connected to the capacitive element (Cᵣ), and a second inductive element (Lₘ) electrically connected to the first inductive element (Lᵣ), wherein the first and second conductive members of the electrosurgical instrument are electrically connected to the second inductive element (Lₘ) in a parallel arrangement, and wherein a resonant frequency (fᵣ) of the arrangement is based on the capacitive element (Cᵣ) and first inductive element (Lᵣ);
selecting an input AC voltage signal to be applied by the input AC voltage source, the selected input AC voltage signal including a selected switching frequency (fₛ) and selected peak voltage value, the switching frequency (fₛ) selected based on the desired V-R characteristic for the target area, the switching frequency (fₛ) selected as a frequency that is greater than the resonant frequency (fᵣ) of the arrangement;
contacting the target area between the first and second conductive members of the electrosurgical instrument; and
while the target area is contacted between the first and second conductive members of the electrosurgical instrument:
applying, by the input AC voltage source, the selected input AC voltage signal having the selected switching frequency (fₛ) and selected peak voltage value;
wherein the configuring of the electrosurgical instrument assembly is performed in such a way that:
when the selected input AC voltage signal having the selected switching frequency (fₛ) and selected peak voltage value is applied by the input AC voltage source at a time t₁, the electrosurgical instrument assembly is configured to apply, via the first and second conductive members of the electrosurgical instrument, an output voltage to the target area that is based on an electrical resistance of the target area at the time t₁; and
when the selected input AC voltage signal having the selected switching frequency (fₛ) and selected peak voltage value continues to be applied by the input AC voltage source after the time t₁, the electrosurgical instrument assembly is configured to apply, via the first and second conductive members of the electrosurgical instrument, an output voltage to the target area that adaptively changes in response to changes in electrical resistance of the target area after the time t₁.

2. The method of claim 1, wherein one or more of the following apply:
the switching frequency (fₛ) is selected in such a way that a ratio of the switching frequency (fₛ) to resonant frequency (fᵣ) is between 1:1 and 2:1; and/or
the configuring of the electrosurgical instrument assembly is performed in such a way that the resonant frequency (fᵣ) is less than or equal to about 1000 kHz and the switching frequency (fₛ) is less than or equal to about 2000 kHz.

3. The method of claim 1, wherein the configuring of the electrosurgical instrument assembly is performed in such a way that the output voltage applied, via the first and second conductive members of the electrosurgical instrument, to the target area after the time t₁ adaptively changes solely in response to changes in electrical resistance of the target area after the time t₁ and without requiring any change to the selected switching frequency (fₛ) and/or selected peak voltage value of the selected input AC voltage signal.

4. The method of claim 3,
wherein the changes in electrical resistance of the target area after the time t₁ are caused by the applying of the selected input AC voltage signal; and
wherein the adaptive changing of the output voltage applied, via the first and second conductive members of the electrosurgical instrument, to the target area after the time t₁, is automatically achieved without any measuring of the actual electrical resistance of the target area.

5. The method of claim 1, wherein the configuring of the electrosurgical instrument assembly is performed in such a way that the output voltage applied, via the first and second conductive members of the electrosurgical instrument, to the target area after the time t₁ adaptively changes solely in response to changes in electrical resistance of the target area after the time t₁ and without requiring any change to the arrangement.

6. The method of claim 1, wherein the plurality of possible electrical resistance values of the target area are electrical resistance values that may exist during an electrosurgical action performed on the target area.

7. The method of claim 6, wherein one or more of the following apply:
the electrosurgical action is a sealing of a vessel; and/or
the target area is a tissue.

8. The method of claim 1, wherein the switching frequency (fₛ) is selected in such a way as to minimize a difference between the output voltage that should be applied to the target area for a particular resistance value pursuant to the desired V-R characteristic and an actual output voltage applied to the target area.

9. The method of claim 8, further comprising associating the actual output voltage applied to the target area with a particular voltage pursuant to the desired V-R characteristic to determine a status of the target area.

10. The method of claim 9, further comprising terminating, by the input AC voltage source, the applying of the selected input AC voltage signal when the actual output voltage applied to the target area reaches a maximum voltage value pursuant to the desired V-R characteristic.

11. The method of claim 1, further comprising sensing at least one parameters of an operating environment, the at least one parameter is selected from a group comprising of temperature, applied pressure, gas formation, or a combination thereof.

12. An electrosurgical system configurable to perform an electrosurgical action on a target area, the electrosurgical system comprising:
an electrosurgical instrument having a first conductive member and a second conductive member, the first and second conductive members configurable to contact with the target area; and
an arrangement of a capacitive element (Cᵣ), a first inductive element (Lᵣ) electrically connected to the capacitive element (Cᵣ), and a second inductive element (Lₘ) electrically connected to the first inductive element (Lᵣ), wherein the first and second conductive members of the electrosurgical instrument are electrically connected to the second inductive element (Lₘ) in a parallel arrangement, wherein a resonant frequency (fᵣ) of the arrangement is based on the capacitive element (Cᵣ) and first inductive element (Lᵣ); and
an input AC voltage source electrically connected to the arrangement, the input AC voltage source configurable to provide an input AC voltage signal having a selected switching frequency (fₛ) and a selected peak voltage value, the switching frequency (fₛ) selected based on a desired V-R characteristic for the target area, the switching frequency (fₛ) selected as a frequency that is greater than the resonant frequency (fᵣ) of the arrangement, wherein the desired V-R characteristic including a plurality of possible electrical resistance values for the target area and a corresponding voltage value to be applied to the target area for each of the possible electrical resistance values;
wherein the arrangement is configured in such a way that:
when a selected input AC voltage signal having the selected switching frequency (fₛ) and selected peak voltage value is applied by the input AC voltage source at a time t₁, the arrangement is configured to apply, via the first and second conductive members of the electrosurgical instrument, an output voltage to the target area that is based on an electrical resistance of the target area at the time t₁; and
when the selected input AC voltage signal having the selected switching frequency (fₛ) and selected peak voltage value continues to be applied by the input AC voltage source after the time t₁, the arrangement is configured to apply, via the first and second conductive members of the electrosurgical instrument, an output voltage to the target area that adaptively changes in response to changes in electrical resistance of the target area after the time t₁.

13. The electrosurgical system of claim 12, wherein one or more of the following apply:
the resonant frequency (fᵣ) is less than or equal to about 1000 kHz and the switching frequency (fₛ) is less than or equal to about 2000 kHz;
the capacitive element (Cᵣ) is less than or equal to about 10 F, the first inductive element (Lᵣ) is less than or equal to about 10 H, and the second inductive element (Lₘ) is less than or equal to about 10 H; and/or
a ratio of the switching frequency (fₛ) to resonant frequency (fᵣ) is between 1:1 and 2:1.

14. The electrosurgical system of claim 12, wherein the output voltage applied, via the first and second conductive members of the electrosurgical instrument, to the target area after the time t₁ adaptively changes solely in response to changes in electrical resistance of the target area after the time t₁ and without requiring any change to the selected switching frequency (fₛ) and/or selected peak voltage value of the selected input AC voltage signal.

15. The electrosurgical system of claim 14,
wherein the changes in electrical resistance of the target area after the time t₁ are caused by the applying of the selected input AC voltage signal; and
wherein the adaptive changing of the output voltage applied, via the first and second conductive members of the electrosurgical instrument, to the target area after the time t₁, is automatically achieved without any measuring of the actual electrical resistance of the target area.

16. The electrosurgical system of claim 12, wherein the output voltage applied, via the first and second conductive members of the electrosurgical instrument, to the target area after the time t₁ adaptively changes solely in response to changes in electrical resistance of the target area after the time t₁ and without requiring any change to the arrangement.

17. The electrosurgical system of claim 12, further comprising a processor, the processor configured to:
receive the desired V-R characteristic for the target area; and
select the switching frequency (fₛ) based on the received desired V-R characteristic for the target area.

18. The electrosurgical system of claim 17, wherein the switching frequency (fₛ) is selected in such a way as to minimize a difference between the output voltage that should be applied to the target area for a particular resistance value pursuant to the desired V-R characteristic and an actual output voltage applied to the target area.

19. The electrosurgical system of claim 18, wherein the processor is further configured to associate the actual output voltage applied to the target area with a particular voltage pursuant to the desired V-R characteristic to determine a status of the target area.

20. The electrosurgical system of claim 18, wherein the processor is further configured to terminate the applying of the selected input AC voltage signal, by the input AC voltage source, when the actual output voltage applied to the target area reaches a maximum voltage value pursuant to the desired V-R characteristic.

21. The electrosurgical system of claim 12, further comprising at least one sensor to sense at least one parameter of an operating environment, the at least one parameter is selected from the group comprising of temperature, applied pressure, gas formation or a combination thereof.

22. The electrosurgical system of claim 12, further comprising a controller, the controller configurable to (1) monitor the output voltage applied to the target area and/or the electrical resistance of the target area, and/or (2) provide control and/or feedback signals to the input AC voltage source.

23. A method of configuring an electrosurgical system, the method comprising:
identifying a target area;
obtaining a desired V-R characteristic for the target area, the desired V-R characteristic including a plurality of possible electrical resistance values for the target area and a corresponding voltage value to be applied to the target area for each of the possible electrical resistance values;
configuring an electrosurgical instrument assembly, the electrosurgical instrument assembly configured to include:
an input AC voltage source configurable to apply an input AC voltage signal, the input AC voltage signal including a switching frequency (fₛ) and a peak voltage value;
an electrosurgical instrument having a first conductive member and a second conductive member; and
an arrangement of a capacitive element (Cᵣ) electrically connected to the input AC voltage source, a first inductive element (Lᵣ) electrically connected to the capacitive element (Cᵣ), and a second inductive element (Lₘ) electrically connected to the first inductive element (Lᵣ), wherein the first and second conductive members of the electrosurgical instrument are electrically connected to the second inductive element (Lₘ) in a parallel arrangement, wherein a resonant frequency (fᵣ) of the arrangement is based on the capacitive element (Cᵣ) and first inductive element (Lᵣ), wherein the resonant frequency (fᵣ) is selected based on the desired V-R characteristic for the target area, and wherein the resonant frequency (fᵣ) is selected as a frequency that is less than the switching frequency (fₛ) of the input AC voltage signal;
contacting the target area between the first and second conductive members of the electrosurgical instrument; and
while the target area is contacted between the first and second conductive members of the electrosurgical instrument:
applying, by the input AC voltage source, the input AC voltage signal;
wherein the configuring of the electrosurgical instrument assembly is performed in such a way that:
when the input AC voltage signal is applied by the input AC voltage source at a time t₁, the electrosurgical instrument assembly is configured to apply, via the first and second conductive members of the electrosurgical instrument, an output voltage to the target area that is based on an electrical resistance of the target area at the time t₁; and
when the input AC voltage signal continues to be applied by the input AC voltage source after the time t₁, the electrosurgical instrument assembly is configured to apply, via the first and second conductive members of the electrosurgical instrument, an output voltage to the target area that adaptively changes in response to changes in electrical resistance of the target area after the time t₁.

24. The method of claim 23, wherein one or more of the following apply:
the resonant frequency (fᵣ) is selected in such a way that a ratio of the switching frequency (fₛ) to resonant frequency (fᵣ) is between 1:1 and 2:1;
the configuring of the electrosurgical instrument assembly is performed in such a way that the resonant frequency (fᵣ) is less than or equal to about 1000 kHz and the switching frequency (fₛ) is less than or equal to about 2000 kHz; and/or
the configuring of the electrosurgical instrument assembly is performed in such a way that the capacitive element (Cᵣ) is less than or equal to about 10 F, the first inductive element (Lᵣ) is less than or equal to about 10 H, and the second inductive element (Lₘ) is less than or equal to about 10 H.

25. The method of claim 23, wherein the configuring of the electrosurgical instrument assembly is performed in such a way that the output voltage applied, via the first and second conductive members of the electrosurgical instrument, to the target area after the time t₁ adaptively changes solely in response to changes in electrical resistance of the target area after the time t₁ and without requiring any change to the selected resonant frequency (fᵣ).

26. The method of claim 23,
wherein the changes in electrical resistance of the target area after the time t₁ are caused by the applying of the input AC voltage signal; and
wherein the adaptive changing of the output voltage applied, via the first and second conductive members of the electrosurgical instrument, to the target area after the time t₁, is automatically achieved without any measuring of the actual electrical resistance of the target area.

27. The method of claim 23, wherein the configuring of the electrosurgical instrument assembly is performed in such a way that the output voltage applied, via the first and second conductive members of the electrosurgical instrument, to the target area after the time t₁ adaptively changes solely in response to changes in electrical resistance of the target area after the time t₁ and without requiring any change to the arrangement.

28. The method of claim 23, wherein the resonant frequency (fᵣ) is selected in such a way as to minimize a difference between the output voltage that should be applied to the target area for a particular resistance value pursuant to the desired V-R characteristic and an actual output voltage applied to the target area.
